(19) United States
(12) Patent Application Publication
(10) Pub. No.: US 2026/0078464 A1
(43) Pub. Date: Mar. 19, 2026

(54) ORE-BREAKER COMPOSITIONS AND METHODS FOR BIOLOGICAL ENRICHMENT OF METALS

(71) Applicant: ECOBIOME HOLDINGS LLC, THE WOODLANDS, TX (US)

(72) Inventor: MARC RODRIGUEZ, THE WOODLANDS, TX (US)

(73) Assignee: ECOBIOME HOLDINGS LLC, THE WOODLANDS, TX (US)

(21) Appl. No.: 18/887,880

(22) Filed: Sep. 17, 2024

(51) Int. Cl. C22B 1/00; C12N 1/20; C12R 1/07

(52) U.S. Cl. CPC C22B 1/00; C12N 1/205; C12R 2001/07

(57) ABSTRACT

Isolated modified Bacillus oredergradus, Bacillus humilyticus, Bacillus metallicyclus, Bacillus chrysorecoverus, Bacillus sulfoassimulus, Bacillus sulfocyclus, Bacillus decompolyticus, Bacillus silicadegradus strains deposited at the Agricultural Research Service Culture Collection under the Accession numbers NRRL No. B-68391, B-68392, B-68393, B-68394, B-68395, B-68396, B-68397, and B-68398 according to the Budapest Treaty. Compositions comprising one or more isolated modified bacterial strains according to the Budapest Treaty. The disclosed newly discovered bacterial strains are useful in gold, silver, and platinum group metal enrichment in ores. In particular, the present disclosure is directed to novel microorganisms useful to enrich these metals in substrates that they are cultured in. Methods of using the novel microorganisms to enrich metals in their ores without reducing pH below 7, and without heating.

### BACKGROUND INFORMATION

### Technical Field

The present disclosure is directed to the field of metals (for example, but not limited to, gold, silver, platinum group metals, lithium, copper, nickel, cobalt, and mixtures thereof) enrichment. In particular, the present disclosure encompasses isolated microorganisms or mixtures thereof, wherein at least some of the microorganisms have been modified relative to wild-type and useful to enrich one or more metals and/or mixtures thereof in a substrate such as an environmental substrate, so that the metals can further be extracted and/or amplified in accordance with our previous patents.

### Background Art

Most current biomining operations target valuable metals like copper, uranium, nickel, and gold that are commonly found in sulfidic (sulfur-bearing) minerals. “What is Biomining?”, American Geoscience Institute (2024). Some microbes oxidize sulfidic minerals, converting metals like iron and copper into forms that can dissolve more easily. Other metals, like gold, are not directly dissolved by this microbial process, but are made more accessible to traditional mining techniques because the minerals surrounding these metals are dissolved and removed by microbial processes. When the metal of interest is directly dissolved, the biomining process is called “bioleaching,” and when the metal of interest is made more accessible or “enriched” in the material left behind, it is called “biooxidation.” Both processes involve microbial reactions that can happen anywhere the microbes, rocks or liquids, and necessary nutrients, like oxygen, occur together, for example, heap leaching, dump leaching, and agitated leaching. The microbes, compositions, and processes of the present disclosure concern biooxidation, making the metals of interest more accessible or “enriched” in the material left behind, especially for gold and other precious metals, such as silver and platinum group metals.

Enriching metals from ores using traditional methods can have several problems, including environmental impact, processing, and waste:
Environmental impact: The extraction process can cause pollution to soil and water and can also tear up the land. This can include deforestation, soil erosion, and loss of wildlife habitats. Heavy equipment can also cause noise pollution, and heavy goods vehicles can release carbon dioxide and other gases into the environment.
Processing: The extraction process can require a lot of energy and can also lead to a large amount of waste material. Other materials, such as fuel, may also be needed to refine the metals.
Complex methods: Some metals may require more complex extraction methods, such as electrolysis, while others may be easier to extract through roasting and smelting.

Copper-sulfide and nickel-sulfide mining is currently used as a primary source of copper and nickel today, and use of microbes is not unknown in the industry. For example, U.S. Pat. Nos. 7,700,343; 7,837,760; 8,372,628; and 10017831 disclose a sulfur-oxidizing bacterium and the use thereof in a bioleaching method at ambient temperatures for sulfured minerals. The bacterium (mesophilic sulfur-oxidizing bacteria) is used in acidic solution to deter the passivation phenomenon that acts as leaching inhibitor. As explained in U.S. Pat. No. 8,163,558 B2, bioleaching is defined as method for solubilizing metals from complex matrixes in an acid medium, employing the direct or indirect action of microorganisms (Rawlings D. E., Microb. Cell Fact. 2005; 4(1):13). It is direct when the microorganisms act on the metal or on its counter ion, releasing an ion of the metal of interest into the solution in both cases. On the other hand, it is indirect when the microorganism does not have either the metal of interest or its counter ion as a substrate but generates chemical conditions that accelerate and favor solubilization of the said metal.

Currently millions of dollars are spent in freighting sulfuric acid to heap leach sites to drop the pH to 1. Also, known methods require heat to be added to the heap. The lowering of pH and increasing temperature by adding heat makes heap leaching very expensive and uneconomical to leach gold and metals. A need in the art exists for microbes, compositions including same, and improved methods of using the microbes and compositions to enrich metals such as gold, platinum group metals, and to a lesser extent lithium, copper, nickel, cobalt, and mixtures thereof.

### SUMMARY

In accordance with the present invention, microbes and compositions are described that may be used to enrich metals in their ores, such as gold, silver, platinum group metals, and to a lesser extent lithium, copper, nickel, cobalt, and mixtures thereof. The novel microbes and compositions of the present disclosure have been proven to oxidize sulfur in ores at neutral/near neutral pH and without added heat to accelerate the reaction, which is advantageous and unexpected. With the microbes, compositions, and methods of the present disclosure, pH and temp are not a factor. The introduction of these microbes now enables sulfur to oxidize at near neutral pH and normal air temperature, resulting in enhanced gold and precious metals recovery without having to invest millions of dollars. One aspect of the present disclosure are compositions comprising an isolated modified bacterial strain Bacillus oredergradus. In certain embodiments, the bacterial strain is ECO006, which has been designated Accession number NRRL No. B-68391.

Another aspect of the present disclosure are compositions comprising an isolated modified bacterial strain Bacillus humilyticus. In certain embodiments, the bacterial strain is ECO007, which has been designated Accession number NRRL No. B-68392.

Another aspect of the present disclosure are compositions comprising an isolated modified bacterial strain Bacillus metallicyclus. In certain embodiments, the bacterial strain is ECO008, which has been designated Accession number NRRL No. B-68393.

One aspect of the present disclosure are compositions comprising an isolated modified bacterial strain Bacillus chrysorecoverus. In certain embodiments, the bacterial strain is ECO009, which has been designated Accession number NRRL No. B-68394.

Another aspect of the present disclosure are compositions comprising an isolated modified bacterial strain Bacillus sulfoassimulus. In certain embodiments, the bacterial strain is ECO010, which has been designated Accession number NRRL No. B-68395.

Another aspect of the present disclosure are compositions comprising an isolated modified bacterial strain Bacillus sulfocyclus. In certain embodiments, the bacterial strain is ECO010, which has been designated Accession number NRRL No. B-68396.

Another aspect of the present disclosure are compositions comprising an isolated modified bacterial strain Bacillus decompolyticus. In certain embodiments, the bacterial strain is ECO012, which has been designated Accession number NRRL No. B-68397.

Another aspect of the present disclosure are compositions comprising an isolated modified bacterial strain Bacillus silicadegradus. In certain embodiments, the bacterial strain is ECO013, which has been designated Accession number NRRL No. B-68398.

All of the above-mentioned bacilli have been deposited in accordance with the Budapest Treaty at the Agricultural Research Service Culture Collection (USDA, ARS, 1815 North University Street, Peoria, IL, 61064) on Jul. 16, 2024.

In another aspect, the disclosure provides a method of enriching gold metal and/or platinum group metals and/or silver metal comprising contacting a solid substrate with a composition comprising an isolated modified bacterial strain selected from Bacillus oredergradus, Bacillus humilyticus, Bacillus metallicyclus, Bacillus chrysorecoverus, Bacillus sulfoassimulus, Bacillus sulfocyclus, Bacillus decompolyticus, and Bacillus silicadegradus, and mixtures of two or more of these. In some embodiments, the method comprises enriching gold metal and the bacterial strain Bacillus oredergradus. In some embodiments, the method comprises enriching gold metal and the bacterial strain Bacillus humilyticus. In some embodiments, the method comprises enriching gold metal and the bacterial strain Bacillus metallicyclus. In some embodiments, the method comprises enriching gold metal and the bacterial strain Bacillus chrysorecoverus. In some embodiments, the method comprises enriching gold metal and the bacterial strain Bacillus sulfoassimulus. In some embodiments, the method comprises enriching gold metal and the bacterial strain Bacillus sulfocyclus. In some embodiments, the method comprises enriching gold metal and the bacterial strain Bacillus decompolyticus. In some embodiments, the method comprises enriching gold metal and the bacterial strain Bacillus silicadegradus. In some embodiments, the composition includes one or more microbes used in industrial mining and as described herein.

In each of the preceding embodiments, the solid substrate may be a geological substrate, such as one or more of sandstone, limestone, shale, coal, chalk deposit formations, refractory rock ore or a solid substrate obtained from one or more of a terrestrial, aquatic or marine source. In each of the preceding embodiments, the solid substrate may be one or more of soil, biofilm, sediment, native metal rock and sludge residue.

In another aspect, the disclosure provides a method of enriching gold metal and/or platinum group metals and/or silver metal from a liquid substrate comprising contacting a liquid substrate with a composition comprising an isolated modified bacterial strain selected from Bacillus oredergradus, Bacillus humilyticus, Bacillus metallicyclus, Bacillus chrysorecoverus, Bacillus sulfoassimulus, Bacillus sulfocyclus, Bacillus decompolyticus, and Bacillus silicadegradus, and mixtures of two or more of these. In some embodiments, the method comprises enriching gold metal and the bacterial strain Bacillus oredergradus. In some embodiments, the method comprises enriching gold metal and the bacterial strain Bacillus humilyticus. In some embodiments, the method comprises enriching gold metal and the bacterial strain Bacillus metallicyclus. In some embodiments, the method comprises enriching gold metal and the bacterial strain Bacillus chrysorecoverus. In some embodiments, the method comprises enriching gold metal and the bacterial strain Bacillus sulfoassimulus. In some embodiments, the method comprises enriching gold metal and the bacterial strain Bacillus sulfocyclus. In some embodiments, the method comprises enriching gold metal and the bacterial strain Bacillus decompolyticus. In some embodiments, the method comprises enriching gold metal and the bacterial strain Bacillus silicadegradus.

In some embodiments, the composition includes one or more microbes used in industrial mining and as described herein.

In some embodiments, the bacterial strain may be inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm (where “CFU” means colony forming units).

In some embodiments, the liquid substrate may be obtained from one or more of a wastewater, sludge water, saltwater, freshwater, irrigation system, pond, lake, river, and estuary source. In each of the preceding embodiments, the substrate may be disinfected and/or sterilized prior to inoculation with the bacterial strain.

In some embodiments, the methods of the present disclosure may further comprise adding a fertilizer, nutrient and/or by product composition one or more times to the substrate after inoculation with the bacterial strain.

In some embodiments, the methods of the present disclosure may include allowing sufficient time for the bacterial strain to colonize and exponentially grow on or in the substrate. As demonstrated in the Examples, “sufficient time” depends primarily on the substrate. In some embodiments, the methods of the present disclosure may comprise using an anodic and cathodic LED having a wavelength generator set at a wavelength range of 2.0-22.0 KHz in the liquid substrate.

In some embodiments, the methods of the present disclosure may comprise harvesting the gold metal and/or platinum group metals and/or silver metal by separating the gold metal and/or platinum group metals and/or silver metal from the substrate.

In another aspect, the disclosure provides compositions comprising an isolated modified bacterial strain selected from Bacillus oredergradus, Bacillus humilyticus, Bacillus metallicyclus, Bacillus chrysorecoverus, Bacillus sulfoassimulus, Bacillus sulfocyclus, Bacillus decompolyticus, and Bacillus silicadegradus, and mixtures of two or more of these, about 0.01%/Wt. gold emulsion, about 0.01%/Wt. gold nanoparticles, about 0.01%/Wt. adenosine triphosphate, about 1%/Wt. seaweed extract, about 0.5%/Wt. humic acid, about 0.5%/Wt. mixture of nitrogen, phosphate, potassium and micronutrient mixture, about 0.1% light and mid chain hydrocarbon mixture, about 0.25%/Wt. green solvent, about 0.75%/Wt. sugar, about 1.0%/Wt. substrate selected from gold, platinum-group metal, or silver, and combinations thereof, and a carrier selected from water, agarose, or mixtures thereof.

In some embodiments, the water carrier may be selected from deionized water, distilled water, filtered water, well water, tap water, fresh water, sea water, brackish water, mineralized water, carbonated water, saline water, ionically charged water, ionized water, hydrogen water and combinations thereof.

In some embodiments, compositions may further comprise one or more components selected from biosolvents ethyl lactate, ATP, ADP, pyrophosphate, soy based solvents, chemical solvents, green solvents, range of organic acids, lactic acid, malic acid, ascorbic acid, alkanes, alkenes, alkynes, saturates, aromatics, resinoids, asphaltenes, light, mid chain and heavy chain hydrocarbons, sodium nitrate, sodium nitrite, ethanol, sulfur, sulfate, sulfite, nitrogen, chemical surfactants (ionic, anionic, cationic, zwitterionic surfactants), and combinations and mixtures thereof, polymers (low, mid, heavy chains), biosurfactants, glycolipids, rhamnolipids (J1 and J2), glycerin, propylene glycol, carbon sugars, dextrose, galactose, sucrose, fructose, complex carbohydrates, starch, cellulose, lignin, keratin, proteins and amino acids, manures, composts, green waste, sludge material, humic and fulvic acids, coal ash and coal derived waste, alumina cytokinins and seaweed extracts.

These and other features of the compositions and methods of the present disclosure are set forth in the description below. Other features or advantages of the present invention will be apparent from the following drawings and detailed description, several examples, and from the appended claims. It should be understood that wherever the term “comprising” is used herein, other embodiments where the term “comprising” is substituted with “consisting essentially of” are explicitly disclosed herein, and vice versa. It should be further understood that wherever the term “comprising” is used herein, other embodiments where the term “comprising” is substituted with “consisting of” are explicitly disclosed herein, and vice versa. Moreover, the use of negative limitations is specifically contemplated; for example, certain compositions may include a surfactant or a certain microbe, while other compositions may be devoid of surfactant or that specific microbe. As another example, certain methods of the present disclosure may comprise a step of adding a fertilizer, nutrient and/or by product composition one or more times to the substrate after inoculation with the bacterial strain; however, certain other method embodiments may be devoid of a step of adding a fertilizer, nutrient and/or by product composition one or more times to the substrate after inoculation with the bacterial strain.

### BRIEF DESCRIPTION OF THE DRAWINGS

The manner in which the objectives of this disclosure and other desirable characteristics can be obtained is explained in the following description and attached drawings in which:

FIGS. 1 and 2 are schematic illustrations of an apparatus useful in discovering and isolating the microorganisms useful in the presently disclosed methods.

### DETAILED DESCRIPTION

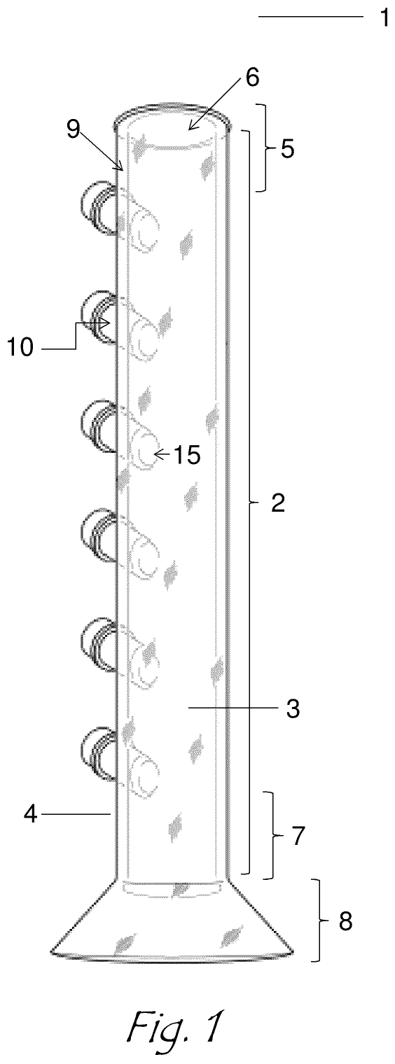

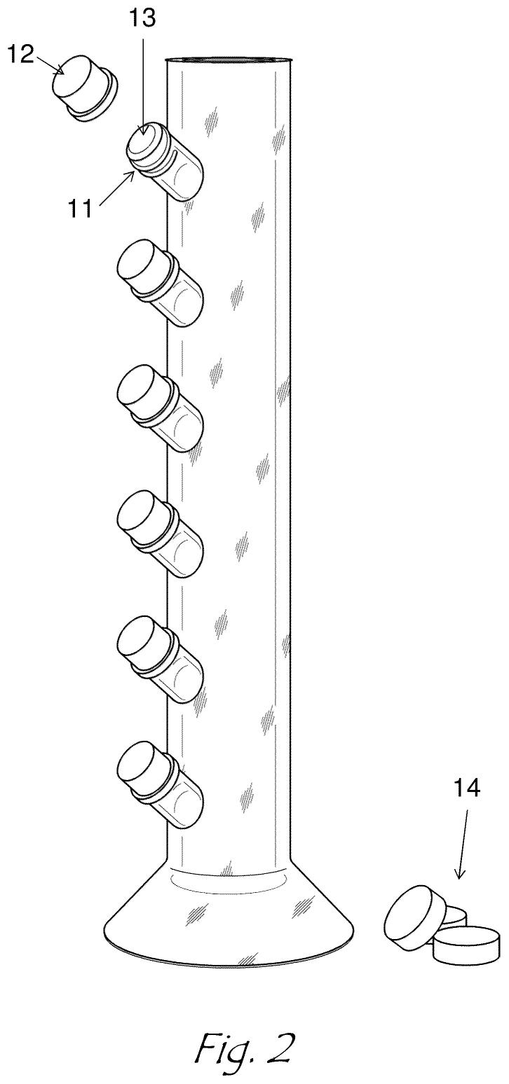

The present disclosure is based, at least in part, on the discovery of eight bacterium isolated from the EcoBiome Innovation and Discovery apparatus, as illustrated schematically in FIGS. 1 and 2. The Ecobiome apparatus and methods of use thereof are described in U.S. Pat. No. 11,845,077 B2 and is herein incorporated by reference in its entirety.

As illustrated schematically in FIG. 1, the apparatus 1 generally comprises a body 2 (e.g., an elongated cylinder) having an interior cavity 3, an exterior 4, an upper end 5, an opening in the upper end configured to receive a sample 6, a closed lower end opposite of the upper end 7, a base attached to the lower end 8, at least one sidewall between the upper end and lower end 9, wherein the at least one sidewall forms the interior cavity configured to retain the sample 3; and at least one lateral port opening attached to the at least one sidewall of the cylinder between the upper end and lower end 10, allowing access into the interior cavity 3 through the internal aperture 13 of the lateral port opening. In preferred embodiments, a plurality of lateral port openings are attached to the at least one sidewall between the upper end and lower end, allowing access to the interior cavity of the apparatus in a variety of positions between the upper and lower ends. The lateral port openings are adapted to allow access to the interior of the cylinder and can be opened and closed, in non-limiting examples by placing a cap, plug, gasket, septum and the like, on the distal end of the lateral port opening. FIG. 2 shows in some embodiments, the distal end of the lateral port opening are threaded 11 as a means to close the distal end with a cap 12. The lateral port opening(s) comprise an internal aperture 13 which is continuous with hole in sidewall 15 at the point of connection with the lateral port, so as to allow access to the interior cavity of the body. FIG. 2 also illustrates exemplary tablets 14 which comprise microorganisms isolated from the apparatus.

Ore samples containing elemental gold (native state gold), and/or gold alloys such as gold alloyed with one or more of copper, palladium, and silver, or gold compounds such as gold tellurides were obtained and prepared for culturing in the Ecobiome apparatus. The ores containing native gold were obtained from a gold mine in Arizona. The mining samples were crushed and loaded into the Ecobiome apparatus for microbial gradation and speciation. The prepped mining sample was allowed to equilibrate, and microbial growth promoted. After some time, a population of microorganisms were isolated, including a newly discovered wild-type bacteria. After isolation, the wild-type bacteria were subjected to a number of extreme culture conditions, such as high and low pH, high and low temperature, and high and low salinity conditions which minimally forced enzymatic change (for example, nitrate reductase, ligninase, cellulase, chitinase, and/or urease) thereby producing a modified bacterial strain relative to wild-type. Thus, as used herein, the term “modified” refers to a bacterial strain that has been forced to change, minimally, with respect to enzymatic gain of function. See, for example, the examples herein.

Modification of the originally isolated wild-type bacteria resulted in the isolation and characterization of ECO006 (Bacillus oredergradus), ECO007 (Bacillus humilyticus), ECO008 (Bacillus metallicyclus), ECO009 (Bacillus chrysorecoverus), ECO010 (Bacillus sulfoassimulus), ECO011 (Bacillus sulfocyclus), ECO012 (Bacillus decompolyticus), ECO013 (Bacillus silicadegradus). Through various characterization methods it was found that each of these facilitated gold metal production, silver metal production, and palladium metal production from a variety of environmental solid and liquid substrata. The isolated bacterial strains Bacillus oredergradus, Bacillus humilyticus, Bacillus metallicyclus, Bacillus chrysorecoverus, Bacillus sulfoassimulus, Bacillus sulfocyclus, Bacillus decompolyticus, and Bacillus silicadegradus demonstrated gold, silver, and palladium production, respectively, de novo, using eco-friendly and sustainable biochemical processes and methods.

Altogether, the present disclosure provides multiple lines of evidence showing the presently disclosed bacterium and methods of using the same to enrich gold, silver, and palladium metal from a variety of environmental substrates. Other aspects and iterations of the invention are described more thoroughly below.

The bacterial strains disclosed in this description have been deposited under conditions that assure that access to the cultures will be available during the pendency of this application. The bacterial strains disclosed in this description have been deposited in the Agricultural Research Service Culture Collection (USDA, ARS, 1815 North University Street, Peoria, Ill., 61064). The bacterial strains deposited were designated as Bacillus oredergradus, Bacillus humilyticus, Bacillus metallicyclus, Bacillus chrysorecoverus, Bacillus sulfoassimulus, Bacillus sulfocyclus, Bacillus decompolyticus, and Bacillus silicadegradus. The deposits were received by the NRRL on Jul. 16, 2024, and were given accession numbers by the International Depository Authority of B-68391, B-68392, B-68393, B68394, B-68395, B-68396, B-68397, and B68398 respectively. The deposits have been made to and received by the International Depository Authority under the provisions of the Budapest Treaty, and all restrictions upon public access to the deposits will be irrevocably removed upon the grant of a patent on this application. The deposits will be available as required by foreign patent laws in countries wherein counterparts of the subject application, or its progeny, are filed. However, it should be understood that the availability of the deposits does not constitute a license to practice the subject invention.

Further, the subject culture deposits will be stored and made available to the public in accord with the provisions of the Budapest Treaty for the Deposit of Microorganisms, i.e., they will be stored with all the care necessary to keep them viable and uncontaminated for a period of at least five years after the most recent request for the furnishing of a sample of a deposit, and in any case, for a period of at least thirty (30) years after the date of deposit or for the enforceable life of any patent which may issue disclosing the cultures. The depositor acknowledges the duty to replace the deposit(s) should the depository be unable to furnish a sample when requested due to the condition of the deposits.

Accordingly, one aspect of the present disclosure encompasses an isolated bacteria strain Bacillus oredergradus (ECO006), Bacillus humilyticus (ECO007), Bacillus metallicyclus (ECO008), Bacillus chrysorecoverus (ECO009), Bacillus sulfoassimulus (ECO010), Bacillus sulfocyclus (ECO011), Bacillus decompolyticus (ECO012), and Bacillus silicadegradus (ECO013), each of which are modified relative to wild-type. Another aspect of the disclosure provides a mutant or derivative of ECO006, ECO007, ECO008, ECO009, ECO010, ECO011, ECO012, or ECO013 having the ability to enrich gold metal, silver metal, or palladium metal as described herein. The term “mutant or derivative” thereof includes naturally occurring and artificially induced mutants which retain their ability to enrich gold metal, silver metal, or palladium metal. Production of such mutants or derivatives will be well known by those skilled in the art including transgenic expression of heterologous nucleic acid sequences and/or genomic modifications.

In another aspect, the present disclosure provides compositions comprising one or more of ECO006, ECO007, ECO008, ECO009, ECO010, ECO011, ECO012, and/or ECO013 or any combination or mixture of these. The concentration(s) of ECO006, ECO007, ECO008, ECO009, ECO010, ECO011, ECO012, and/or ECO013 will vary depending on the type of composition. For example, but not being limited thereto, suitable ECO006 concentrations include but are not limited to at least about 0.5×10¹⁰ CFU/Gm, least about 1.0×10¹⁰ CFU/Gm, least about 1.5×10¹⁰ CFU/Gm, least about 2.0×10¹⁰ CFU/Gm, least about 2.5×10¹⁰ CFU/Gm, least about 3.0×10¹⁰ CFU/Gm, least about 3.5×10¹⁰ CFU/Gm, least about 4.0×10¹⁰ CFU/Gm, least about 4.5×10¹⁰ CFU/Gm, least about 5.0×10¹⁰ CFU/Gm or greater. Compositions where ECO007, ECO008, ECO009, ECO010, ECO011, ECO012, or ECO013 is substituted for ECO006 at these aforementioned concentrations are also contemplated.

A composition comprising ECO006, or ECO007, or ECO008, or ECO009, or ECO010, or ECO011, or ECO012 and/or ECO013 or any combination or mixture of these, according to the present disclosure may comprise one or more additional components, including but not limited to, biosolvents ethyl lactate, ATP, ADP, pyrophosphate, soy based solvents, chemical solvents, green solvents, range of organic acids, lactic acid, malic acid, ascorbic acid, alkanes, alkenes, alkynes, saturates, aromatics, resinoids, asphaltenes, light, mid chain and heavy chain hydrocarbons, sodium nitrate, sodium nitrite, ethanol, sulfur, sulfate, sulfite, nitrogen, chemical surfactants (ionic, anionic, cationic, zwitterionic surfactants), polymers (low, mid, heavy chains), biosurfactants, glycolipids, rhamnolipids (J1 and J2), glycerin, propylene glycol, carbon sugars, dextrose, galactose, sucrose, fructose, complex carbohydrates, starch, cellulose, lignin, keratin, proteins and amino acids, fertilizer NPK (for example, organic and inorganic fertilizers), manures, composts, green waste, sludge material, humic and fulvic acids, coal ash and coal derived waste, alumina cytokinins and seaweed extracts.

A composition comprising ECO006, or ECO007, or ECO008, or ECO009, or ECO010, or ECO011, or ECO012 and/or ECO013, or any combination or mixture of these, according to the present disclosure may comprise a water source for microbial culturing or final product carrier. Non-limiting examples include deionized water, distilled water, filtered water, well water, tap water, fresh water, sea water, brackish water, mineralized water, carbonated water, saline water, ionically charged water, ionized water, and hydrogen water. Thus, according to the present disclosure compositions comprising microorganisms of the disclosure for use within the methods of the disclosure may comprise a water source for microbial culturing or final product carrier. Non-limiting examples include deionized water, distilled water, filtered water, well water, tap water, fresh water, sea water, brackish water, mineralized water, carbonated water, saline water, ionically charged water, ionized water, and hydrogen water. The aqueous solution may contain sufficient nutrients to support microbial growth. The useful nutrients are both inorganic and organic compounds commonly used to grow and nourish microbes. Inorganic nutrients include nitric acid, ammonium nitrate, ammonium chloride, ammonium sulfate, sodium nitrate, sulfur, sodium sulfide, sodium chloride, sodium bicarbonate, sodium phosphate, potassium phosphate, sulfuric acid, nitric acid, cyanide, uranium, mercury, lead, lithium, sodium metabisulfite, ammonium nitrate, fertilizers, gluconic acid, phosphogypsum, ferric chloride, calcium chloride, and ammonium phosphate. Organic nutrients include microbial biomass, glucose, dextrose, sodium acetate, amino acids, and purines. Vitamins that can be included in the nutrient solution include pyridoxine, pyridoxamine-HCl, riboflavin, thiamine, niacin, pantothenic acid, p-aminobenzoic acid, folic acid, and biotin. Small amounts of trace elements such as iron, copper, molybdenum and zinc can also be provided in the nutrient solution. Useful nutrients can also be mineral ores used for recovery of metals.

A composition comprising ECO006, or ECO007, or ECO008, or ECO009, or ECO010, or ECO011, or ECO012 and/or ECO013, or any combination or mixture of these according to the present disclosure may be formulated as a soil mixture, liquid, sludge or slurry substrate.

In some embodiments, a composition comprising ECO006, or ECO007, or ECO008, or ECO009, or ECO010, or ECO011, or ECO012 and/or ECO013, or any combination or mixture of these according to the present disclosure may comprise sulfuric acid, nitric acid, cyanide, uranium, mercury, lead, lithium, sodium metabisulfite, ammonium nitrate, fertilizers, gluconic acid, or phosphogypsum.

In one aspect, an ECO006 composition of the present disclosure may comprise Bacillus oredergradus at a concentration of about 2.5×10¹⁰ CFU/Gm (5.0%/Wt.), gold emulsions at a concentration of about 0.01%/Wt., gold nanoparticles at a concentration of about 0.01%/Wt., adenosine Triphosphate (ATP) at a concentration of about 0.01%/Wt., seaweed at a concentration of about 1%/Wt., humic acid at a concentration of about 0.5%/Wt., NPK and micronutrients at a concentration of about 0.5%/Wt., a mixture of light and mid chain hydrocarbons at a concentration of about 0.1%/Wt., green solvents at a concentration of about 0.25%/Wt., a carbon sugar source at a concentration of about 0.75%/Wt., a representative substrate at a concentration of about 1.0%/Wt.; and an inert carrier, for example, a liquid or agarose which makes up the remainder of the mass to 100% final Weight (%/Wt.). Compositions where ECO007, or ECO008, or ECO009, or ECO010, or ECO011, or ECO012 and/or ECO013 is substituted for ECO006 at these aforementioned concentrations are also contemplated.

Another aspect of the present disclosure is a method to extract, produce and/or amplify gold metal, silver metal, and/or platinum group metals comprising culturing suitable microbe or plurality of suitable microbes. Non-limiting examples of suitable microbes include acidophilic archaea such as Sulfolobus metallicus and Metallosphaera sedula; mesophilic bacteria of the genera Acidithiobacillus or Leptospirillum ferrooxidans; Pyrococcus furiosus; thermoacidophilic archaeon Sulfolobus (Metallosphaera sedula); and Pyrobaculum islandicum. These microorganisms are basically 10, belonging to Bacteria: Acidiphilium sp., Leptospirillum sp., Sulfobacillus sp., Acidithiobacillus ferrooxidans and Acidithiobacillus thiooxidans; and Archaea: Acidianus sp., Ferroplasma sp., Metallosphaera sp., Sulfolobus sp. and Thermoplasma sp.

In a preferred embodiment, the methods include the bacterium strain ECO006 or a mutant or derivative thereof; the bacterium strain ECO007 or a mutant or derivative thereof; the bacterium strain ECO008 or a mutant or derivative thereof; the bacterium strain ECO009 or a mutant or derivative thereof; the bacterium strain ECO010 or a mutant or derivative thereof; the bacterium strain ECO011 or a mutant or derivative thereof; the bacterium strain ECO012 or a mutant or derivative thereof; or the bacterium strain ECO013 or a mutant or derivative thereof.

Without wishing to be bound by theory, the mode of action of producing gold, silver, and platinum group metals is due to ECO006, ECO007, ECO008, ECO009, ECO009, ECO010, ECO011, ECO012, and ECO013's innate ability to enrich gold, silver, platinum group metals so as to render them more accessible to other microbes, such as disclosed in our pending U.S. patent application Ser. No. 18/254,461, filed May 25, 2023 (isolated modified bacterial strain Thiomonas isabelensis, which has been designated Accession number NRRL No. B-67995, deposited in accordance with the Budapest Treaty at the Agricultural Research Service Culture Collection (USDA, ARS, 1815 North University Street, Peoria, IL, 61064) on Nov. 13, 2020). The isolated modified bacterial strain Thiomonas isabelensis is able to extract, produce and/or amplify the metals, respectively from the environmental substrate that it is cultured in. Isolated Thiomonas isabelensis is found to express various proteins and biochemicals which are modulated by the base concentrations of metals in their surrounding environments. Additionally, ECO006, ECO007, ECO008, ECO009, ECO009, ECO010, ECO011, ECO012, and ECO013 each generate a high concentration of biomass, or microbial colonies throughout the culturing process, that enable them to hold high rates of gold, silver, and platinum group metals within their respective intracellular matrix.

For purposes of this disclosure, the term “mineral” or “mineral ore” means a composition that comprises gold, silver, and/or platinum group metal values. Thus, a mineral may be a mined mineral, ancient seabed deposit, ancient lakebed deposit, black sands, an ore concentrate, metal bearing sea water, and waste products, such as mining tails, industrial waste water, oil well brine, coal tars, oil shales, tar sands, and oil sands. Useful minerals contain trace amounts of gold, silver, and/or platinum group metals. Trace amount means the detection limit or below detection limits of conventional assay procedures such as fire assay, AAS (atomic adsorption spectroscopy), ICP-MS (inductive coupled plasma-mass spectrometer), ICP-AES (atomic emission spectroscopy) and other spectroscopic instrumentation commonly used in analytical laboratories. Some spectroscopic methods can detect as little as 1 ppt (part per trillion) to 0.1 ppb (part per billion).

In some embodiments, the methods of enriching gold, silver, and/or platinum group metals generally comprise farming the gold, silver, and/or platinum group metals including the steps of inoculating the bacterial strains disclosed herein on solid substrates or geological substrates. A geologic substrate is a surface (or volume) of sediment or rock where physical, chemical, and biological processes occur, such as the movement and deposition of sediment, the formation of bedforms, and the attachment, burrowing, feeding, reproduction, and sheltering of organisms. Non limiting examples of a geological substrate useful for the present disclosure include sandstone, limestone, shale, coal, chalk deposit formations, refractory rock ore (for example, single, double and triple refractory rock ore). Additional solid substrates include but are not limited to an environmental sample collected from any terrestrial, aquatic or marine source such as soil, biofilms, sediments (for example, coral or other marine sediments, aquifer sediments and the like), native metal rocks and sludge residue. In some embodiments, the solid substrate is disinfected prior to inoculation of the bacterial strains disclosed herein. Disinfection techniques include but are not limited to steam, autoclave, oven, microwave, biocide and fungicide solutions. Additional substrates include but are not limited to animal manures, bauxite, base metals, calcium phosphate, calcium silicate, clays and silicates, aluminum oxide, diatomaceous earth, diammonium phosphate, erionite and zeolites, feldspar, flint, food wastes, granite, gypsum, humic and fulvic acids, marble, mica, molten rock and lava, monoammonium phosphate, potash, pumice, silica, slate, seaweed, talc and recycled electronics and commercial devices.

In some embodiments, one or more of ECO006, ECO007, ECO008, ECO009, ECO009, ECO010, ECO011, ECO012, and/or ECO013 may be applied to any solid substrate in a rock, powder, granulated or broken form for improved gold metal, silver metal, and/or platinum group metal biooxidation and enrichment. In some embodiments, solid substrates are used in traditional farming, specialty farming, potted and greenhouse farming, hydroponics and aeroponics techniques. In some embodiments, the solid substrate includes old or recycled electronic components or batteries.

In one embodiment, the biological enrichment processes using microbes according to the disclosure is conducted in commercially available bioreactor consisting of a reactor having an agitation means. The agitation means can be mechanical stirring with a flat bladed impeller, percolation column, or air agitated pachuca reactor. The bioreactor can have air intake means, sterilization means, harvesting means, heating and/or cooling means, temperature controller means, pH controller means, filtration means and pressure controller means. All these features of bioreactors are known and commercially available in the biotechnology industry.

The biological enrichment processes using microbes according to the disclosure can also be done prior to or simultaneous with heap bioleaching techniques. In heap bioleaching techniques, a large body of mineral ore is treated with mutant microbes in nutrient solution in large contaminant ponds with no agitation and/or only occasional agitation. Generally, the contact time for heap type biotreatment is substantially longer than the agitated bioreactors and range from 10 days to 100 days.

As used herein, the term “inoculating” refers to the act of introducing a microorganism or a plurality of microorganisms (for example ECO006, ECO007, ECO008, ECO009, ECO009, ECO010, ECO011, ECO012, and/or ECO013) into a substrate where it will be metabolically active and/or propagate. In preferred embodiments, the step of inoculating is performed using aseptic technique. In some embodiments, the bacterial strain is inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm. In a preferred embodiment, the bacterial strain is inoculated at a concentration from about 1.0×10⁶ CFU/gm to about 1.0×10¹² CFU/gm. The inoculating step can occur one or more times during the duration of enriching gold, silver, and/or platinum metal from the solid substrate.

Inoculation of the solid substrate can occur by any means known to the skilled artisan which provides the microbe to the substrate in a sufficient amount. In some embodiments, after inoculation additional solid substrate is added to increase the surface area of the solid substrate which is in contact with the bacterial strain. In one aspect, additional solid substrate is added to create a 4-6 inch depth over the initial inoculation depth.

After inoculation the solid substrate is optionally irrigated and/or fertilized one or more times to stimulate colonization and exponential growth throughout of the bacterial strain throughout the solid substrate. Exemplary fertilizers include a low NPK plus micronutrient fertilizer, solutions comprising a complex or simple sugar, a seaweed or cytokine and a vitamin blend. In addition, specialty nutrients and by-products can be added to the inoculated solid substrate one or more times to establish new, increased and rigorous colonization by the bacterial strain.

In addition, the solid substrate may be covered to maintain a stable temperature or allow for an increase in the solid substrate temperature, for example using a poly covering for consistent temperature control and to control microbial contaminants from colonizing. In some embodiments, the inoculated solid substrate is maintained at a temperature between about 20° C. to about 60° C. including any range therein. In a preferred embodiment, the inoculated solid substrate is maintained at a temperature between about 29° C. to about 50° C.

After the bacterial strain has had sufficient time to colonize and biochemically process the solid substrate (non-limiting example 2-10 weeks) the solid substrate can be tested and processed for gold, silver, and/or platinum group metals production, and/or subjected to further processing in the presence of an extraction or amplifying microbe such as Thiomonas isabelensis. The testing and/or processing steps include harvesting the solid substrate which has been colonized by the bacterial strain, generating a slurry by adding a solution to the solid substrate, and centrifugation at a minimum of 8,000 RPM to concentrate the precipitate which contains the de novo gold, silver, and/or platinum group metals. These steps may optionally include a bacterial lysis step to release any metals within the bacterial strains intracellular matrix.

In a still another aspect, the methods of enriching gold, silver, and/or platinum group metals generally comprise inoculating the bacterial strains disclosed herein in liquid substrates. Suitable liquid substrates include but are not limited to balanced salt and nutrient solutions, broths, environmental samples collected from any aquatic or marine source, waste waters, sludge waters, saltwater, freshwater, irrigation systems, ponds, lakes, rivers, and estuaries. In some embodiments, the liquid substrate is disinfected prior to inoculation of the bacterial strains disclosed herein. Disinfection techniques include but are not limited to steam, autoclave, oven, microwave, biocide and fungicide solutions. In a preferred embodiment, the disinfection step will reduce microbial colony and propagule concentrations to below or at about 5.0×10⁵ CFU/ml.

Inoculation of the liquid substrate can occur by any means known to the skilled artisan at concentration described above for the solid substrate. The inoculating step can occur one or more times during the duration of enriching gold, silver, and/or platinum group metals from the liquid substrate. After inoculation the liquid substrate is preferably agitated during the enrichment of the gold, silver, and/or platinum group metals. In an exemplary embodiment, agitation of the liquid substrate occurs using an air pump for aerobic respiration.

After inoculation of the liquid substrate, optionally specialty nutrients and by-products can be added to the inoculated liquid substrate one or more times to establish new, increased and rigorous colonization by the bacterial strain, for example by adding solutions comprising a complex or simple sugar, a seaweed or cytokinin and a vitamin blend. In addition, for accelerated reactions establish an anodic and cathodic LED using a wavelength generator set at a range of 2.0-22.0 KHz.

After the bacterial strain has had sufficient time to colonize and biochemically process the liquid substrate (non-limiting example 12-72 hours) the liquid substrate can be tested and processed for gold, silver, and/or platinum group metals production and/or subjected to further processing in the presence of an extraction or amplifying microbe such as Thiomonas isabelensis. The testing and/or processing steps include collecting the liquid substrate which has been colonized by the bacterial strain or strains, generating a slurry by adding a solution to the solid substrate, and centrifugation of the liquid solution through an in line and continuous centrifuge at a minimum of 8,000 RPM to concentrate the precipitation. These steps may optionally include a bacterial lysis step to release any metals within the bacterial strains intracellular matrix.

In each of the above embodiments, a bioreactor, fermenter, reaction vessel can be used in the disclosed methods. Moreover, the present disclosure contemplates the use of the disclosed microbes before or during bioleaching and heap leaching and therefore the use of leach pits are contemplated within the methods as well.

Bio treatment temperature ranges from 15 degrees centigrade to 50 degrees centigrade, preferably from 20 degrees to 30 degrees centigrade. pH can be acidic (pH 1 to 3) or basic (pH 9 to 12), although slightly acidic (pH 4) to slightly basic (pH 8) pH ranges are preferred. The most preferred pH ranges are the neutral range of from pH 6.5 to pH 7.5.

In accordance with the methods of the present disclosure, pressure is not critical and can be at atmospheric, below atmospheric, and/or above atmospheric. The biological transmutation process can be conducted in aerobic or anaerobic conditions. The biological transmutation process can be conducted in the presence of nitrogen, carbon dioxide, and oxygen in the atmosphere. Oxygen can be provided chemically, for example, with hydrogen peroxide, or as a gas from pressurized vessels.

Microbe concentration is not critical. At low microbe concentration, the contact duration is generally longer to allow the microbe to grow and multiply. However, microbe concentration should not exceed the maximum microbe concentration that the nutrient solution can sustain. Contact time can vary from a few hours to several weeks and depends in part on the type and mesh size of the mineral ore in which enrichment is occurring. Contact time ranges can be from 1 day to 30 days, more preferably from 1 day to 10 days.

The biological process using microbes according to the disclosure can be conducted in aerobic or anaerobic conditions. However, they are preferably conducted in the presence of oxygen, nitrogen and carbon dioxide in the atmosphere. Oxygen can also be provided chemically, for example, with hydrogen peroxide, or as a gas from pressurized vessels.

Nutrients can also be provided during the biological transmutation process to support growth of the mutant microbes. Nutrients can be inorganic, including nitric acid, sulfur, ammonium nitrate, ammonium chloride, ammonium sulfate, sodium nitrate, sodium chloride, sodium bicarbonate, sodium phosphate, potassium nitrate, potassium phosphate, ferric chloride, calcium chloride, and ammonium phosphate, and organic, including glucose, dextrose, sodium acetate, amino acids, and purines. Vitamins that can be included in the nutrient solution include pyridoxine, pyridoxamine-HCl, riboflavin, thiamine, niacin, pantothenic acid, p-aminobenzoic acid, folic acid, and biotin. Small amounts of traces elements such as iron, copper, molybdenum and zinc can also be provided in the nutrient solution.

After the biooxidation (enrichment) process, the resulting composition maybe subjected to further biological amplification using microbes including, but not limited to, Thiomonas isabelensis, and/or recovery of metal produced from the starting material and microbial solution can be performed by conventional metallurgical methods such as smelting, leaching, electrolysis, resins and other methods known to those skilled in art of metallurgy. In another embodiment, the precious metals in the microbes or biomass of dead microbes can be recovered by methods described for recovery of precious metals from mineral ore.

Fire assaying and cupellation are described by C. W. Ammen, Recovery and Refining of Precious Metals, second edition 1993, Chapter 12, pp 302-329.

Also provided are kits. Such kits can include an agent or composition described herein and, in certain embodiments, instructions for use. Such kits can facilitate performance of the methods described herein. When supplied as a kit, the different components of the composition can be packaged in separate containers and admixed immediately before use. Components include but are not limited to ECO006, ECO007, ECO008, ECO009, ECO009, ECO010, ECO011, ECO012, and/or ECO013 compositions and formulations for use or stability, as described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile agarose each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988, Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligomicleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); and B. Perbal, A practical Guide To Molecular Cloning (1984); F. M. Ausubel et al. (eds.).

So that the present disclosure may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, and the like. This same principle applies to ranges reciting only one numerical value as a minimum or a maximum. Furthermore, such an interpretation should apply regardless of the breadth of the range or the characteristics being described.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

When introducing elements of the present disclosure or the preferred aspects(s) thereof, the articles “a,” “an,” “the,” and “said” are intended to mean that there are one or more of the elements unless the contrary is explicitly mentioned. The terms “comprising,” “including,” and “having” are intended to be inclusive and mean that there may be additional elements other than the listed elements. Negative limitations are also contemplated by the present disclosure; for example, a composition may be devoid of Bacillus oredergradus; a method may be devoid of one or more steps.

In this disclosure, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open-ended term, like “comprising” or “including,” it is understood that direct support should be afforded also to “consisting essentially of” language as well as “consisting of” language as if stated explicitly and vice versa.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### Example 1: Novel Microorganism Isolation and Classification

It has been estimated that only 2% of all microbial isolates can be cultured in a lab. Therefore, the microbes that can be grown in the laboratory represent only a small fraction of the total diversity that exists in nature. At all levels of microbial phylogeny, uncultured clades that do not grow on standard media are playing critical roles in cycling carbon, nitrogen, and other elements, synthesizing novel by-products, and impacting the surrounding organisms and environment. The ability to culture difficult to culture or previously uncultured microbial strains provides a wealth of information about their role in the environment, ecology, and nutrient cycling. But perhaps even more importantly, screening of novel isolates will reveal novel products that can have profound effects for the discovery of novel drugs, improve agricultural techniques and products, and in the production of copper, cobalt, nickel, and other metals. To solve this problem the Applicant developed a novel apparatus (Ecobiome Discovery apparatus) allowing for the controlled growth, isolation and characterization of microorganisms including those that are difficult to culture or are uncultivable at the present time.

In an effort to isolate and characterize novel microbes, an experiment utilizing the Ecobiome Discovery apparatus was used. Ores were obtained and prepared for culturing in the Ecobiome apparatus. Double refractory ores containing native gold were obtained from an underground Arizona gold mine. In short, in each case the sample was crushed and loaded into the Ecobiome apparatus for passive microbial gradiation and speciation. The prepped samples were allowed to equilibrate, and microbial growth promoted. After some time, a population of microorganisms, including the newly discovered organisms (ECO006, ECO007, ECO008, ECO009, ECO009, ECO010, ECO011, ECO012, and/or ECO013) were isolated.

After isolation of the individual microbe populations comprising ECO006, ECO007, ECO008, ECO009, ECO009, ECO010, ECO011, ECO012, and/or ECO013, challenge tests were performed on each, which included tests specific for gold enrichment microbes. These tests resulted in the isolation and characterization of ECO006 (Bacillus oredergradus), ECO007 (Bacillus humilyticus), ECO008 (Bacillus metallicyclus), ECO009 (Bacillus chrysorecoverus), ECO010 (Bacillus sulfoassimulus), ECO011 (Bacillus sulfocyclus), ECO012 (Bacillus decompolyticus), and ECO013 (Bacillus silicadegradus).

ECO006, ECO007, ECO008, ECO009, ECO010, ECO011, ECO012, and ECO013 are each gram-negative bacteria, non-spore formers that are capable of metabolizing simple and complex polymers as well as metals through heterotrophic and chemoheterotrophic biochemical pathways. ECO006, ECO007, ECO008, ECO009, ECO010, ECO011, ECO012, and ECO013 are facultative anaerobes and are tolerant of low and high temperatures, for example, ranging between about 5° C. to about 46° C. In addition, each is tolerant of a pH range from about 2 to about 9. Each microorganism reaches exponential growth with high agitation (>360 rpm) and oxygenation (DO>90%) within 6-8 hours.

TABLE 1
EcoBiome microorganism and biome analysis comparison

| | Untreated | Bio- | Minimum |
|---|---|---|---|
| | As is | Treated | Threshold |
| Microorganism | (CFU/Gm) | (CFU/Gm) | (CFU/Gm) |
| Sulfur cycling microbes | 7,000 | 84,000 | 55,000 |
| Sulfur oxidizing microbes | 13,500 | 93,000 | 70,000 |
| Carbon cycling microbes | 4,400 | 102,000 | 75,000 |
| Humic acid decomposing microbes | 17,100 | 99,000 | 55,000 |
| Fulvic acid decomposing microbes | 8.900 | 110,000 | 60,000 |
| Black lead cycling microbes | 3,400 | 89,000 | 70,000 |

### Example 2: Enriching Gold by Decreasing Non-Gold Species in Ore

Five pounds of a double refractory gold ore sample from the same Arizona mine was used for each trial. The five pounds were weighed out and placed in a bucket container fashioned specifically for holding soil and dry material during processing in a bioreactor. The bucket of ore was inserted into the bioreactor and the reactor was filled to 40 gallons of filtered water. Once the water level exceeded the height of the bucket the circulating water pump was turned on in addition to a continuous air pump into the bioreactor. The EcoBiome ECO006 microorganism (Bacillus oredergradus) was transferred and inoculated into the bioreactor at a rate of 1.0×10¹⁰ CFU/ml. Nutrient contents were then added which consisted of a blend of NPK fertilizer, seaweed extract, zinc citrate, zinc sulfate, Vitamin blend, Dextrose and 3 liters of Nutrient Broth (autoclaved and sterilized). The reaction was closed and sealed and allowed to react for 96 hours with an internal liquid temperature of approximately 40° C. using continuous aeration and recirculation at 350 rpm agitation. (Note that sulfur oxidation occurred at 22° C.) The pH was about 7. Once the reaction was completed, all liquid contents were run through centrifugation, harvested, dried in an oven and then analyzed via an X-Ray Fluorescence Analyzer for non-gold species. The % reduction in the non-gold ore species is reported in Table 2 for Heap Leach Column Tests using X-ray Fluorescence testing (XRF).

TABLE 2
Arizona Gold Mine: Double Refractory Gold Ore Analysis

| Leach Column Analysis | Untreated (As is) | Bio-Treated |
|---|---|---|
| Total Carbon (%) | 1.67 | 0.21 |
| Carbon Carbonate (%) | 0.24 | 0.02 |
| Organic Carbon (%) | 0.93 | 0.11 |
| Total Sulfur (%) | 10.35 | 2.55 |
| Sulfur Sulfate (%) | 4.55 | 1.89 |
| Sulfide (%) | 5.81 | 2.57 |
| pH | 2 | 7 |

As can be seen from Tables 1-2, the bio-treated double refractory gold ore sample with ECO006, inoculation exhibited significant decrease in concentration of the species indicated in Table 2, meaning that the Ecobiome treated samples all showed greater concentrations of gold.

Comparative Assays and Enzymatic Activity Tests-Wild types vs. inventive microorganisms

### Description of Tests

### Microbial Urease Enzyme Activity Assay

This assay measures the activity of the Urease enzyme. Urease is a protein enzyme that catalyzes the hydrolysis of urea into NH₃ and CO₂ according to the following reaction:

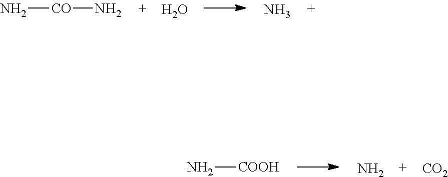

### Microbial Respiration Assay

This assay measures the ability of a microorganism to utilize oxygen or an alternative final electron acceptor in its metabolic pathway.

### Microbial Phosphorus Solubilization Assay

This assay measures the ability of a microorganism to cleave phosphorus from a compound and then metabolize that compound.

### Microbial Hydrogen Sulfide Assay

This assay measures the ability of a microorganism to metabolize sulfur compounds and produce H₂S and gas.

### Microbial Chitinase Enzyme Assay

This assay measures the ability of a microorganism to produce an extracellular Chitinase enzyme that catabolizes chitin compounds in the soil.

### Microbial Cellulase Enzyme Activity

This assay measures the ability of a microorganism to produce an extracellular Cellulase enzyme that catabolizes cellulose and hemi-cellulose compounds in the soil.

### Microbial Ligninase Enzyme Activity

This assay measures the ability of a microorganism to produce an extracellular Ligninase enzyme that catabolizes lignin compounds in the soil.

### Humic Acid Decomposition Enzyme Activity

This test measures the ability of a microorganism to produce an extracellular enzyme to decompose humic acid.

### Fulvic Acid Decomposition Enzyme Activity

This test measures the ability of a microorganism to produce an extracellular enzyme to decompose fulvic acid.

### Sulfur Oxidation Enzyme Activity

This test measures the ability of a microorganism to produce an extracellular enzyme to oxidize sulfur.

Each of microorganisms ECO006, ECO007, ECO008, ECO009, ECO010, ECO011, ECO012, and ECO013 were tested and compared to their wild types according to these ten tests. Results of these ten comparative tests are listed in Tables 3-12.

TABLE 3
Urease Enzyme Activity

| Microorganism | Urease Enzyme Activity |
|---|---|
| wild type 1 | − |
| Bacillus oredergradus ECO006 mutated | ++ |
| wild type 2 | − |
| Bacillus humilyticus ECO007 mutated | ++ |
| wild type 3 | − |
| Bacillus metallicyclus ECO008 mutated | + |
| wild type 4 | + |
| Bacillus chrysorecoverus ECO009 mutated | ++ |
| wild type 5 | − |
| Bacillus sulfoassimulus ECO010 mutated | ++ |
| wild type 6 | − |
| Bacillus sulfocyclus ECO011 mutated | ++ |
| wild type 7 | − |
| Bacillus decompolyticus ECO012 mutated | ++ |
| wild type 8 | − |
| Bacillus silicadegradus ECO013 mutated | ++ |

| | |
|---|---|
| Legend | |
| − | No Enzymatic Activity |
| + | Enzymatic Activity |
| ++ | Strong Enzymatic Activity |

Results for Table 3: All microbial strains were able to effectively degrade urea-based fertilizers for enhanced nitrogen uptake.

TABLE 4
Bacterial Final Electron Acceptor Metabolic Activity

| Microorganism | Metabolic Oxygen Activity |
|---|---|
| wild type 1 | ++ |
| Bacillus oredergradus ECO006 mutated | − |
| wild type 2 | + |
| Bacillus humilyticus ECO007 mutated | − |
| wild type 3 | ++ |
| Bacillus metallicyclus ECO008 mutated | − |
| wild type 4 | + |
| Bacillus chrysorecoverus ECO009 mutated | − |
| wild type 5 | ++ |
| Bacillus sulfoassimulus ECO010 mutated | − |
| wild type 6 | + |
| Bacillus sulfocyclus ECO011 mutated | − |
| wild type 7 | ++ |
| Bacillus decompolyticus ECO012 mutated | − |
| wild type 8 | + |
| Bacillus silicadegradus ECO013 mutated | − |

Legend

| | |
|---|---|
| Legend | |
| − | No Enzymatic Activity |
| + | Enzymatic Activity |
| ++ | Strong Enzymatic Activity |

Results for Table 4: All microorganisms were able to effectively grow and thrive at the roots of the plants with minimal oxygen for enhanced plant growth promotion.

TABLE 5
Phosphorus Solubilization Activity

| | Phosphorous Enzyme |
|---|---|
| Microorganism | Activity |
| wild type 1 | − |
| Bacillus oredergradus ECO006 mutated | ++ |
| wild type 2 | − |
| Bacillus humilyticus ECO007 mutated | ++ |
| wild type 3 | − |
| Bacillus metallicyclus ECO008 mutated | ++ |
| wild type 4 | − |
| Bacillus chrysorecoverus ECO009 mutated | ++ |
| wild type 5 | − |
| Bacillus sulfoassimulus ECO010 mutated | ++ |
| wild type 6 | − |
| Bacillus sulfocyclus ECO011 mutated | ++ |
| wild type 7 | − |
| Bacillus decompolyticus ECO012 mutated | ++ |
| wild type 8 | − |
| Bacillus silicadegradus ECO013 mutated | ++ |

| | |
|---|---|
| Legend | |
| − | No Enzymatic Activity |
| + | Enzymatic Activity |
| ++ | Strong Enzymatic Activity |

Results for Table 5: All microbial strains were effectively able to degrade and release phosphorus from the soil for enhanced phosphorus plant root uptake.

TABLE 6
Hydrogen Sulfide Activity (post 2-weeks)

| Microorganism | Hydrogen Sulfide Activity |
|---|---|
| wild type 1 | − |
| Bacillus oredergradus ECO006 mutated | + |
| wild type 2 | − |
| Bacillus humilyticus ECO007 mutated | ++ |
| wild type 3 | − |
| Bacillus metallicyclus ECO008 mutated | + |
| wild type 4 | − |
| Bacillus chrysorecoverus ECO009 mutated | ++ |
| wild type 5 | − |
| Bacillus sulfoassimulus ECO010 mutated | ++ |
| wild type 6 | − |
| Bacillus sulfocyclus ECO011 mutated | ++ |
| wild type 7 | − |
| Bacillus decompolyticus ECO012 mutated | ++ |
| wild type 8 | + |
| Bacillus silicadegradus ECO013 mutated | ++ |

| | |
|---|---|
| Legend | |
| − | No Enzymatic Activity |
| + | Enzymatic Activity |
| ++ | Strong Enzymatic Activity |

Results for Table 6: All microorganisms did not produce the hydrogen sulfide in a low oxygen environment, this were able to effectively metabolize nutrients with substitute electron acceptors besides oxygen for efficient plant growth promotion.

TABLE 7
Chitinase Enzyme Activity

| Microorganism | Chitinase Enzyme Activity |
|---|---|
| wild type 1 | − |
| Bacillus oredergradus ECO006 mutated | ++ |
| wild type 2 | − |
| Bacillus humilyticus ECO007 mutated | ++ |
| wild type 3 | − |
| Bacillus metallicyclus ECO008 mutated | + |
| wild type 4 | − |
| Bacillus chrysorecoverus ECO009 mutated | ++ |
| wild type 5 | − |
| Bacillus sulfoassimulus ECO010 mutated | ++ |
| wild type 6 | − |
| Bacillus sulfocyclus ECO011 mutated | ++ |
| wild type 7 | − |
| Bacillus decompolyticus ECO012 mutated | ++ |
| wild type 8 | − |
| Bacillus silicadegradus ECO013 mutated | ++ |

| | |
|---|---|
| Legend | |
| − | No Enzymatic Activity |
| + | Enzymatic Activity |
| ++ | Strong Enzymatic Activity |

Results for Table 7: All microorganisms were able to degrade and decompose chitin, the second most abundant complex polymer in the soil and the primary component of fungal walls and nematode eggs, for improved soil mineralization.

TABLE 8
Cellulase Enzyme Activity

| Microorganism | Cellulase Enzyme Activity |
|---|---|
| wild type 1 | − |
| Bacillus oredergradus ECO006 mutated | ++ |
| wild type 2 | − |
| Bacillus humilyticus ECO007 mutated | ++ |
| wild type 3 | − |
| Bacillus metallicyclus ECO008 mutated | + |
| wild type 4 | − |
| Bacillus chrysorecoverus ECO009 mutated | ++ |
| wild type 5 | − |
| Bacillus sulfoassimulus ECO010 mutated | ++ |
| wild type 6 | − |
| Bacillus sulfocyclus ECO011 mutated | ++ |
| wild type 7 | − |
| Bacillus decompolyticus ECO012 mutated | ++ |
| wild type 8 | − |
| Bacillus silicadegradus ECO013 mutated | ++ |

| | |
|---|---|
| Legend | |
| − | No Enzymatic Activity |
| + | Enzymatic Activity |
| ++ | Strong Enzymatic Activity |

Results for Table 8: All microbial strains were able to effectively degrade and decompose cellulose, the most abundant complex polymer in the soil for increased mineralization and nutrient cycling. Cellulose is the building block of all stalk, stover and crop residue.

TABLE 9
Ligninase Enzyme Activity

| Microorganism | Ligninase Enzyme Activity |
|---|---|
| wild type 1 | − |
| Bacillus oredergradus ECO006 mutated | ++ |
| wild type 2 | − |
| Bacillus humilyticus ECO007 mutated | ++ |
| wild type 3 | − |
| Bacillus metallicyclus ECO008 mutated | ++ |
| wild type 4 | + |
| Bacillus chrysorecoverus ECO009 mutated | ++ |
| wild type 5 | − |
| Bacillus sulfoassimulus ECO010 mutated | ++ |
| wild type 6 | − |
| Bacillus sulfocyclus ECO011 mutated | ++ |
| wild type 7 | − |
| Bacillus decompolyticus ECO012 mutated | ++ |
| wild type 8 | − |
| Bacillus silicadegradus ECO013 mutated | ++ |

| | |
|---|---|
| Legend | |
| − | No Enzymatic Activity |
| + | Enzymatic Activity |
| ++ | Strong Enzymatic Activity |

Results for Table 9: All microbial strains were able to effectively degrade and decompose lignin, one of the most abundant complex polymers in trees for increased mineralization and nutrient cycling.

TABLE 10
Humic Acid Decomposition Enzyme Activity

| | Humic Acid Decomposition |
|---|---|
| Microorganism | Enzyme Activity |
| wild type 1 | − |
| Bacillus oredergradus ECO006 mutated | ++ |
| wild type 2 | − |
| Bacillus humilyticus ECO007 mutated | ++ |
| wild type 3 | − |
| Bacillus metallicyclus ECO008 mutated | + |
| wild type 4 | − |
| Bacillus chrysorecoverus ECO009 mutated | ++ |
| wild type 5 | − |
| Bacillus sulfoassimulus ECO010 mutated | ++ |
| wild type 6 | − |
| Bacillus sulfocyclus ECO011 mutated | ++ |
| wild type 7 | − |
| Bacillus decompolyticus ECO012 mutated | ++ |
| wild type 8 | − |
| Bacillus silicadegradus ECO013 mutated | ++ |

| | |
|---|---|
| Legend | |
| − | No Enzymatic Activity |
| + | Enzymatic Activity |
| ++ | Strong Enzymatic Activity |

Results for Table 10: All microbial strains were able to effectively degrade and decompose humic acid, one of the most abundant sources of carbon in nature and ore.

TABLE 11
Fulvic Acid Decomposition Enzyme Activity

| | Fulvic Acid Decomposition |
|---|---|
| Microorganism | Enzyme Activity |
| wild type 1 | − |
| Bacillus oredergradus ECO006 mutated | ++ |
| wild type 2 | − |
| Bacillus humilyticus ECO007 mutated | ++ |
| wild type 3 | − |
| Bacillus metallicyclus ECO008 mutated | ++ |
| wild type 4 | + |
| Bacillus chrysorecoverus ECO009 mutated | ++ |
| wild type 5 | − |
| Bacillus sulfoassimulus ECO010 mutated | ++ |
| wild type 6 | − |
| Bacillus sulfocyclus ECO011 mutated | ++ |
| wild type 7 | − |
| Bacillus decompolyticus ECO012 mutated | ++ |
| wild type 8 | − |
| Bacillus silicadegradus ECO013 mutated | ++ |

| | |
|---|---|
| Legend | |
| − | No Enzymatic Activity |
| + | Enzymatic Activity |
| ++ | Strong Enzymatic Activity |

Results for Table 11: All microbial strains were able to effectively degrade and decompose fulvic acid, one of the most abundant sources of carbon in nature and ore.

TABLE 12
Sulfur Oxidation Enzyme Activity

| | Fulvic Acid Decomposition |
|---|---|
| Microorganism | Enzyme Activity |
| wild type 1 | − |
| Bacillus oredergradus ECO006 mutated | ++ |
| wild type 2 | − |
| Bacillus humilyticus ECO007 mutated | ++ |
| wild type 3 | − |
| Bacillus metallicyclus ECO008 mutated | ++ |
| wild type 4 | − |
| Bacillus chrysorecoverus ECO009 mutated | ++ |
| wild type 5 | − |
| Bacillus sulfoassimulus ECO010 mutated | ++ |
| wild type 6 | − |
| Bacillus sulfocyclus ECO011 mutated | ++ |
| wild type 7 | − |
| Bacillus decompolyticus ECO012 mutated | ++ |
| wild type 8 | − |
| Bacillus silicadegradus ECO013 mutated | ++ |

| | |
|---|---|
| Legend | |
| − | No Enzymatic Activity |
| + | Enzymatic Activity |
| ++ | Strong Enzymatic Activity |

Results for Table 12: All microbial strains were able to effectively degrade and decompose sulfur, one of the most abundant elements in nature and ores.

Tables 13-15 summarize further test results.

TABLE 13
Diverse Phosphorus and Sulfur Sources in Nature

| Microorganism | A | B | C | D | E | F |
|---|---|---|---|---|---|---|
| wild type 1 | − | − | − | − | − | |
| Bacillus oredergradus ECO006 mutated | ++ | ++ | ++ | ++ | ++ | ++ |
| wild type 2 | − | − | − | − | − | − |
| Bacillus humilyticus ECO007 mutated | ++ | ++ | ++ | ++ | ++ | ++ |
| wild type 3 | − | − | − | − | − | − |
| Bacillus metallicyclus ECO008 mutated | ++ | ++ | ++ | ++ | ++ | ++ |
| wild type 4 | − | − | − | − | − | − |
| Bacillus chrysorecoverus ECO009 mutated | ++ | ++ | ++ | ++ | ++ | ++ |
| wild type 5 | − | − | − | − | − | − |
| Bacillus sulfoassimulus ECO010 mutated | ++ | ++ | ++ | ++ | ++ | ++ |
| wild type 6 | − | − | − | − | − | − |
| Bacillus sulfocyclus ECO011 mutated | ++ | ++ | ++ | ++ | ++ | ++ |
| wild type 7 | − | − | − | − | − | − |
| Bacillus decompolyticus ECO012 mutated | ++ | ++ | ++ | ++ | ++ | ++ |
| wild type 8 | − | − | − | − | − | − |
| Bacillus silicadegradus ECO013 mutated | ++ | ++ | ++ | ++ | ++ | ++ |

Legend
− No Metabolic Activity
+ Metabolic Activity
++ Strong Metabolic Activity
A = phosphate
B = pyrophosphate
C = Inositol hexa-phosphate
D = Sulfate
E = Cysteine (amino acid)
F = Methionine (amino acid)

TABLE 14
Diverse Saline and Osmotic Conditions in Nature and Ore

| Microorganism | G | H | I | J | K | L |
|---|---|---|---|---|---|---|
| wild type 1 | − | − | − | − | − | |
| Bacillus oredergradus ECO006 mutated | ++ | ++ | ++ | ++ | ++ | ++ |
| wild type 2 | − | − | − | − | − | − |
| Bacillus humilyticus ECO007 mutated | ++ | + | ++ | ++ | ++ | ++ |
| wild type 3 | − | − | − | − | − | − |
| Bacillus metallicyclus ECO008 mutated | ++ | + | ++ | ++ | ++ | ++ |
| wild type 4 | − | − | − | − | − | − |
| Bacillus chrysorecoverus ECO009 mutated | ++ | ++ | ++ | ++ | ++ | ++ |
| wild type 5 | − | − | − | − | − | − |
| Bacillus sulfoassimulus ECO010 mutated | ++ | ++ | ++ | ++ | ++ | ++ |
| wild type 6 | − | − | − | − | − | − |
| Bacillus sulfocyclus ECO011 mutated | ++ | ++ | ++ | ++ | ++ | ++ |
| wild type 7 | − | − | − | − | − | − |
| Bacillus decompolyticus ECO012 mutated | ++ | ++ | ++ | ++ | ++ | ++ |
| wild type 8 | − | − | − | − | − | − |
| Bacillus silicadegradus ECO013 mutated | ++ | ++ | ++ | ++ | ++ | ++ |

Legend
− No Metabolic Activity
+ Metabolic Activity
++ Strong Metabolic Activity
G = NaCl 6%/wt.
H = KCl 6%/wt.
I = Urea 6%/wt.
J = Ammonium sulfate pH 8, 100 mM
K = Sodium nitrate pH 8, 100 mM
L = NaCl 6%/wt. + β Glutamic Acid

TABLE 15
Diverse Saline and Osmotic Conditions in Nature and Ore

| | | pH | | | | pH |
|---|---|---|---|---|---|---|
| | pH | 4.5 + | pH | pH | pH | 9.5 + |
| Microorganism | 4 | Urea | 7 | 9 | 9.5 | Urea |
| wild type 1 | − | − | − | − | − | |
| Bacillus oredergradus | ++ | ++ | ++ | ++ | ++ | ++ |
| ECO006 mutated | | | | | | |
| wild type 2 | − | − | − | − | − | |
| Bacillus humilyticus | ++ | ++ | ++ | ++ | ++ | ++ |
| ECO007 mutated | | | | | | |
| wild type 3 | − | − | − | − | − | − |
| Bacillus metallicyclus | ++ | ++ | ++ | ++ | ++ | ++ |
| ECO008 mutated | | | | | | |
| wild type 4 | − | − | − | − | − | − |
| Bacillus chrysorecoverus | ++ | ++ | ++ | ++ | ++ | ++ |
| ECO009 mutated | | | | | | |
| wild type 5 | − | − | − | − | − | − |
| Bacillus sulfoassimulus | ++ | ++ | ++ | ++ | ++ | ++ |
| ECO010 mutated | | | | | | |
| wild type 6 | − | − | − | − | − | − |
| Bacillus sulfocyclus | ++ | ++ | ++ | ++ | ++ | ++ |
| ECO011 mutated | | | | | | |
| wild type 7 | − | − | − | − | − | − |
| Bacillus decompolyticus | ++ | ++ | ++ | ++ | ++ | ++ |
| ECO012 mutated | | | | | | |
| wild type 8 | − | − | − | − | − | − |
| Bacillus silicadegradus | ++ | ++ | ++ | ++ | ++ | ++ |
| ECO013 mutated | | | | | | |

Legend
− No Metabolic Activity
+ Metabolic Activity
++ Strong Metabolic Activity

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or” as used herein in the specification and in the claims, should be understood to mean “either or both” of the elements so conjoined, in other words, elements that are conjunctively present in some cases and disjunctively present in other cases. Multiple elements listed with “and/or” should be construed in the same fashion, in other words, “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); and the like.

As used herein in the specification and in the claims, “or” should be understood to have the same meaning as “and/or” as defined above. For example, when separating items in a list, “or” or “and/or” shall be interpreted as being inclusive, in other words, the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (in other words “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. through human intervention.

What is claimed is:

1. An isolated bacterial strain Bacillus oredergradus, which has been designated Accession number NRRL No. B-68391, deposited in accordance with the Budapest Treaty at the Agricultural Research Service Culture Collection (USDA, ARS, 1815 North University Street, Peoria, IL, 61064) on Jul. 16, 2024.

2. A method of enriching gold, silver, and/or platinum group metals comprising inoculating a substrate selected from solid substrates, geological substrates, and liquid substrates with the isolated bacterial strain of claim 1.

3. The method of claim 2, wherein the bacterial strain is inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm.

4. The method of claim 2, wherein the solid substrate is obtained from one or more of a terrestrial, aquatic or marine source.

5. The method of claim 4, wherein, the solid substrate is one or more of soil, biofilm, sediment, native metal rock and sludge residue.

6. The method of claim 2, wherein the geological substrate is one or more of sandstone, limestone, shale, coal, chalk deposit formations, refractory rock ore.

7. An isolated bacterial strain Bacillus humilyticus which has been designated Accession number NRRL No. B-68392, deposited in accordance with the Budapest Treaty at the Agricultural Research Service Culture Collection (USDA, ARS, 1815 North University Street, Peoria, IL, 61064) on Jul. 16, 2024.

8. A method of enriching gold, silver, and/or platinum group metal comprising inoculating a substrate selected from solid substrates, geological substrates, and liquid substrates with the isolated bacterial strain of claim 7.

9. The method of claim 8, wherein the solid substrate is obtained from one or more of a terrestrial, aquatic or marine source.

10. The method of claim 9, wherein, the solid substrate is one or more of soil, biofilm, sediment, native metal rock and sludge residue.

11. The method of claim 8, wherein the geological substrate is one or more of sandstone, limestone, shale, coal, chalk deposit formations, refractory rock ore.

12. An isolated bacterial strain Bacillus metallicyclus, which has been designated Accession number NRRL No. B-68393, deposited in accordance with the Budapest Treaty at the Agricultural Research Service Culture Collection (USDA, ARS, 1815 North University Street, Peoria, IL, 61064) on Jul. 16, 2024.

13. A method of enriching gold, silver, and/or platinum group metal comprising inoculating a substrate selected from solid substrates, geological substrates, and liquid substrates with the isolated bacterial strain of claim 12.

14. The method of claim 13, wherein the bacterial strain is inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm.

15. The method of claim 13, wherein the solid substrate is obtained from one or more of a terrestrial, aquatic or marine source.

16. The method of claim 13, wherein, the solid substrate is one or more of soil, biofilm, sediment, native metal rock and sludge residue.

17. The method of claim 13, wherein the geological substrate is one or more of sandstone, limestone, shale, coal, chalk deposit formations, refractory rock ore.

18. An isolated bacterial strain Bacillus chrysorecoverus, which has been designated Accession number NRRL No. B-68394, deposited in accordance with the Budapest Treaty at the Agricultural Research Service Culture Collection (USDA, ARS, 1815 North University Street, Peoria, IL, 61064) on Jul. 16, 2024.

19. A method of enriching gold, silver, and/or platinum group metals comprising inoculating a substrate selected from solid substrates, geological substrates, and liquid substrates with the isolated bacterial strain of claim 18.

20. The method of claim 19, wherein the bacterial strain is inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm.

21. The method of claim 19, wherein the solid substrate is obtained from one or more of a terrestrial, aquatic or marine source.

22. The method of claim 19, wherein, the solid substrate is one or more of soil, biofilm, sediment, native metal rock and sludge residue.

23. The method of claim 19, wherein the geological substrate is one or more of sandstone, limestone, shale, coal, chalk deposit formations, refractory rock ore.

24. An isolated bacterial strain Bacillus sulfoassimulus which has been designated Accession number NRRL No. B-68395, deposited in accordance with the Budapest Treaty at the Agricultural Research Service Culture Collection (USDA, ARS, 1815 North University Street, Peoria, IL, 61064) on Jul. 16, 2024.

25. A method of enriching gold, silver, and/or platinum group metal comprising inoculating a substrate selected from solid substrates, geological substrates, and liquid substrates with the isolated bacterial strain of claim 24.

26. The method of claim 25, wherein the solid substrate is obtained from one or more of a terrestrial, aquatic or marine source.

27. The method of claim 26, wherein, the solid substrate is one or more of soil, biofilm, sediment, native metal rock and sludge residue.

28. The method of claim 25, wherein the geological substrate is one or more of sandstone, limestone, shale, coal, chalk deposit formations, refractory rock ore.

29. An isolated bacterial strain Bacillus sulfocyclus, which has been designated Accession number NRRL No. B-68396, deposited in accordance with the Budapest Treaty at the Agricultural Research Service Culture Collection (USDA, ARS, 1815 North University Street, Peoria, IL, 61064) on Jul. 16, 2024.

30. A method of enriching gold, silver, and/or platinum group metal comprising inoculating a substrate selected from solid substrates, geological substrates, and liquid substrates with the isolated bacterial strain of claim 29.

31. The method of claim 30, wherein the bacterial strain is inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm.

32. The method of claim 30, wherein the solid substrate is obtained from one or more of a terrestrial, aquatic or marine source.

33. The method of claim 32, wherein, the solid substrate is one or more of soil, biofilm, sediment, native metal rock and sludge residue.

34. The method of claim 30, wherein the geological substrate is one or more of sandstone, limestone, shale, coal, chalk deposit formations, refractory rock ore.

35. An isolated bacterial strain Bacillus decompolyticus which has been designated Accession number NRRL No. B-68397, deposited in accordance with the Budapest Treaty at the Agricultural Research Service Culture Collection (USDA, ARS, 1815 North University Street, Peoria, IL, 61064) on Jul. 16, 2024.

36. A method of enriching gold, silver, and/or platinum group metal comprising inoculating a substrate selected from solid substrates, geological substrates, and liquid substrates with the isolated bacterial strain of claim 35.

37. The method of claim 36, wherein the solid substrate is obtained from one or more of a terrestrial, aquatic or marine source.

38. The method of claim 37, wherein, the solid substrate is one or more of soil, biofilm, sediment, native metal rock and sludge residue.

39. The method of claim 36, wherein the geological substrate is one or more of sandstone, limestone, shale, coal, chalk deposit formations, refractory rock ore.

40. An isolated bacterial strain Bacillus silicadegradus, which has been designated Accession number NRRL No. B-68398, deposited in accordance with the Budapest Treaty at the Agricultural Research Service Culture Collection (USDA, ARS, 1815 North University Street, Peoria, IL, 61064) on Jul. 16, 2024.

41. A method of enriching gold, silver, and/or platinum group metal comprising inoculating a substrate selected from solid substrates, geological substrates, and liquid substrates with the isolated bacterial strain of claim 40.

42. The method of claim 41, wherein the bacterial strain is inoculated at a concentration of about 1.0×10³ CFU/gm, about 1.0×10⁴ CFU/gm, about 1.0×10⁴ CFU/gm, about 1.0×10⁶ CFU/gm, about 1.0×10⁷ CFU/gm, about 1.0×10⁸ CFU/gm, about 1.0×10⁹ CFU/gm, about 1.0×10¹⁰ CFU/gm, about 1.0×10¹¹ CFU/gm, about 1.0×10¹² CFU/gm, about 1.0×10¹³ CFU/gm, about 1.0×10¹⁴ CFU/gm, about 1.0×10¹⁵ CFU/gm, about 1.0×10¹⁶ CFU/gm, or about 1.0×10¹⁷ CFU/gm.

43. The method of claim 41, wherein the solid substrate is obtained from one or more of a terrestrial, aquatic or marine source.

44. The method of claim 43, wherein, the solid substrate is one or more of soil, biofilm, sediment, native metal rock and sludge residue.

45. The method of claim 41, wherein the geological substrate is one or more of sandstone, limestone, shale, coal, chalk deposit formations, refractory rock ore.

46. A composition comprising:
a) an isolated bacterial strain selected from the group consisting of:
i) an isolated bacterial strain Bacillus oredergradus, which has been designated Accession number NRRL No. B-68391;
ii) an isolated bacterial strain Bacillus humilyticus which has been designated Accession number NRRL No. B-68392;
iii) an isolated bacterial strain Bacillus metallicyclus, which has been designated Accession number NRRL No. B-68393;
iv) an isolated bacterial strain Bacillus chrysorecoverus, which has been designated Accession number NRRL No. B-68394;
v) an isolated bacterial strain Bacillus sulfoassimulus which has been designated Accession number NRRL No. B-68395;
vi) an isolated bacterial strain Bacillus sulfocyclus, which has been designated Accession number NRRL No. B-68396;
vii) an isolated bacterial strain Bacillus decompolyticus which has been designated Accession number NRRL No. B-68397; and
viii) Bacillus silicadegradus, which has been designated Accession number NRRL No. B-68398,
wherein all of (i)-(viii) were deposited in accordance with the Budapest Treaty at the Agricultural Research Service Culture Collection (USDA, ARS, 1815 North University Street, Peoria, IL, 61064) on Jul. 16, 2024;
b) about 0.01%/Wt. gold emulsion;
c) about 0.01%/Wt. gold nanoparticles;
d) about 0.01%/Wt. adenosine triphosphate;
e) about 1%/Wt. seaweed extract;
f) about 0.5%/Wt. humic acid;
g) about 0.5%/Wt. mixture of nitrogen, phosphate, potassium and micronutrient mixture;
h) about 0.1% light and mid chain hydrocarbon mixture;
i) about 0.25%/Wt. green solvent;
j) about 0.75%/Wt. sugar;
k) about 1.0%/Wt. substrate comprising gold; and
l. a carrier selected from water or agarose.

47. The method in accordance with any of claims 2, 8, 13, 19, 25, 30, 36, and 41 devoid of any step to lower pH below 7 and devoid of any heating.
